(19) United States
(12) Patent Application Publication
(10) Pub. No.: US 2012/0277798 A1
(43) Pub. Date: Nov. 1, 2012

(54) Spinal Rod Construct to Limit Facet Impingement

(75) Inventors: Nicholas Benson, Cordova, TN (US); Christopher Nelson, Collierville, TN (US)

(73) Assignee: WARSAW ORTHOPEDIC, INC., Warsaw, IN (US)

(21) Appl. No.: 13/096,604

(22) Filed: Apr. 28, 2011

(51) Int. Cl. A61B 17/70; A61B 17/56

(52) U.S. Cl. 606/264; 606/278; 606/86 A

(57) ABSTRACT

A spinal rod includes an elongate member and an end feature. The elongate member has a diameter. The end feature is configured to be selectively connected to the elongate member. The end feature includes an extending member that projects radially away from the elongate member. The radial extent has an outer distance greater than the diameter of the elongate member.

### FIELD OF INVENTION

Embodiments of the invention relate to spinal fixation systems. More particularly, the embodiments relate to spinal rods for use in spinal fixation systems.

### BACKGROUND

The spinal column is a biomechanical structure composed primarily of support structures including vertebrae and intervertebral discs and soft tissue structures for motive and stabilizing forces including muscles and ligaments. The biomechanical functions of the spinal column include support, spinal cord protection, and motion control between the head, trunk, arms, pelvis, and legs. These biomechanical functions may require oppositely designed structures. For example, the support function may be best addressed with rigid load bearing structures while motion control may be best suited for structures that are easily movable relative to each other. The trade-offs between these biomechanical functions may be seen within the structures that make up the spinal column. Damage to one or more components of the spinal column, such as an intervertebral disc, may result from disease or trauma and cause instability of the spinal column and damage multiple biomechanical functions of the spinal column. To prevent further damage and overcome some of the symptoms resulting from a damaged spinal column, a spinal fixation device may be installed to stabilize the spinal column.

A spinal fixation device generally consists of stabilizing elements, such as rods or plates, attached by anchors to the vertebrae in the section of the vertebral column that is to be stabilized. The spinal fixation device restricts the movement of the vertebrae relative to one another and supports at least a part of the stresses created by the weight of the body otherwise imparted to the vertebral column. Typically, the stabilizing element is rigid and inflexible and is used in conjunction with an intervertebral fusion device to promote fusion between adjacent vertebral bodies. There are some disadvantages associated with the use of rigid spinal fixation devices, including decreased mobility, stress shielding (i.e. too little stress on some bones, leading to a decrease in bone density), and stress localization (i.e. too much stress on some bones, leading to fracture and other damage).

In a common spinal system, screws are fixed in bone. The screws have a receiver head generally comprising a U-shaped channel for receiving a spinal rod. Extenders from the receiver channel may guide the rod as it is delivered to toward the screw head. A set screw may hold the rod within the receiver when the spinal rod is fully seated in the receiver. The set screw may lock the rod to the screw.

Additionally, effects may be realized on the vertebral levels adjacent the spinal fixation device. The construct may extend into the space of the adjacent vertebral body, which may limit motion at that level. For example, the rod may extend into the facet area between levels and cause impingement of the facet joint. This may be particularly true in a medialized construct, where the system is placed in cortical bone located close to the spinal processes of the vertebra. Thus, it would be beneficial to design a spinal rod that may limit interaction with adjacent vertebral levels.

The description herein of problems and disadvantages of known apparatuses, methods, and devices is not intended to limit the invention to the exclusion of these known entities. Indeed, embodiments of the invention may include, as a part of the embodiment, portions or all of one or more of the known apparatus, methods, and devices without suffering from the disadvantages and problems noted herein.

### SUMMARY OF THE INVENTION

An embodiment of the invention may include a spinal rod having an elongate member and an end feature. The elongate member has a diameter. The end feature is configured to be selectively connected to the elongate member. The end feature includes an extending member that projects radially away from the elongate member. The radial extent has an outer distance greater than the diameter of the elongate member.

Another embodiment of the invention may include a spinal construct including a spinal rod, a screw assembly and a set screw. The spinal rod may include an elongate member and an end feature. The elongate member has a diameter. The end feature is configured to be selectively connected to the elongate member. The end feature includes an extending member that projects radially away from the elongate member. The radial extent has an outer distance greater than the diameter of the elongate member. The screw assembly may have a U-shaped channel for receiving the elongate member and a bone engaging portion for attaching to bone. The U-shaped channel may have a pair of arms extending away from the bone engaging portion. The arms are configured to mate with a portion of the end feature to limit the movement of the elongate member relative to the screw assembly. The set screw is configured to capture the elongate member to the screw assembly.

Additional aspects and features of the present disclosure will be apparent from the detailed description and claims as set forth below.

### BRIEF DESCRIPTION OF THE DRAWINGS

FIG. 1 is a view of a spinal rod according to an embodiment of the present invention.

FIG. 2 is a view of the elongate member of FIG. 1.

FIG. 3 is a view of the end ring of FIG. 1.

FIG. 4 is a view of a spinal rod according to another embodiment of the invention.

FIG. 5 is a view of the spinal rod of FIG. 1 attached to two screw assemblies.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

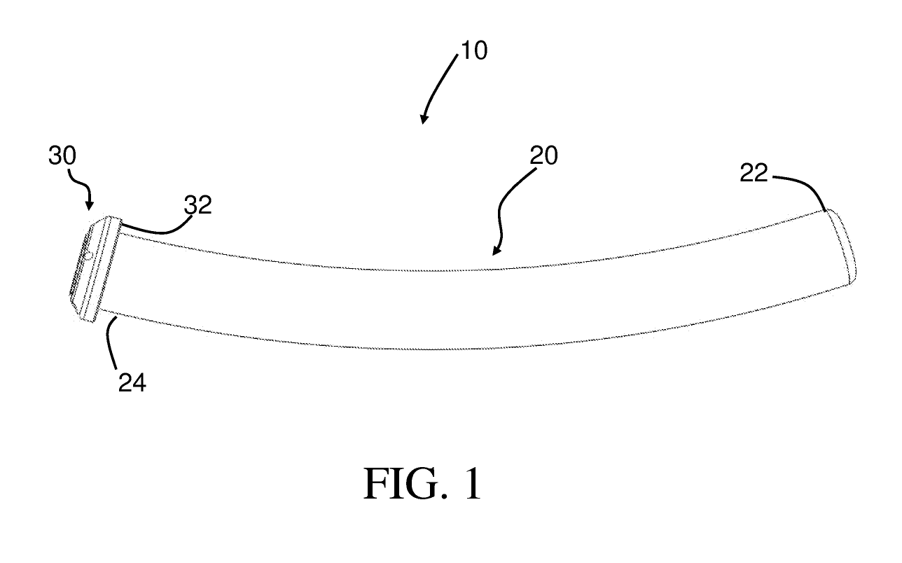

Turning now to FIG. 1, view of a spinal rod 10 according to an embodiment of the present invention. The spinal rod 10 includes an elongate member 20 and an end feature, which in this embodiment is an end ring 30. The elongate member 20 may be a curved rod or a straight rod. The elongate member 20 may be fixed to bone anchoring members, such as screws or hooks, to fix the spinal rod 10 to the spinal column. The length of the elongate member 20 is determined by the number of levels the surgeon desires the rod to span. The diameter of the elongate member 20, as well as the material of the elongate member 20 determines the stiffness of the spinal rod 10 and the spinal construct.

The end ring 30 may be attached to the end of the elongate member 30 in order to guide the rod into a screw assembly. For example, if screw extenders project upward from a screw, the end ring 30 may contact the screw extenders and slide along the extenders into contact with the screw. A contact surface 32 on the end ring 30 may provide a stop for the spinal rod 20 so that the spinal rod 20 may not be pulled through the screw assembly. The contact surface 32 may be a planar surface that may allow the spinal rod 20 to abut the sides of a screw assembly. In other embodiments, the contact surface 32 may be tapered or notched to allow for the spinal rod 20 to snugly fit against the screw assembly.

The end ring 30 allows for minimal overhang of the spinal rod 20 when fixed to a spinal screw assembly. In prior art systems, the spinal rod extends outside the screw assembly to make the implant easier. For example, the surgeon may distract the spinal column as he fixes the screws to the spinal rod. The extension of the rod past the ends of the screw assemblies allows the rod to slide within the screw assembly without disengaging the screw assembly. Because the distraction may linearly adjust the rod from either side, the overhang must be present on both sides. Thus, the overhang extends superiorly and inferiorly over the cephalic and caudal levels, respectively. The present embodiment allows for one portion of the rod 20 to be linearly limited. In most instances, the cephalic level will be linearly limited. Such a configuration allows impingement to be avoided superiorly, where the surgeon likely wishes to maintain as much freedom for motion as possible. However, an embodiment where the end ring 30 is located inferiorly on the spinal rod 20 may be useful in certain spinal constructs. Yet another embodiment may allow for the end ring 30 to be positioned on both ends of the elongate member 20.

The end ring 30 may be fixed to the elongate member 20 after the elongate member 20 is placed in the screw assemblies. This may be beneficial, for example, in a percutaneous or mini-open procedure. In these procedures, the elongate member 20 may be delivered to the screw assemblies subcutaneously. Such procedures may limit the amount of muscle and soft tissue retraction that must be performed in order to place the elongate member 20 in the screw assemblies. Once the elongate member 20 is placed into the receiver of the screw assembly, the end ring 30 may be delivered to the end of the elongate member 20 and attached to the elongate member 20 to form the spinal rod 10.

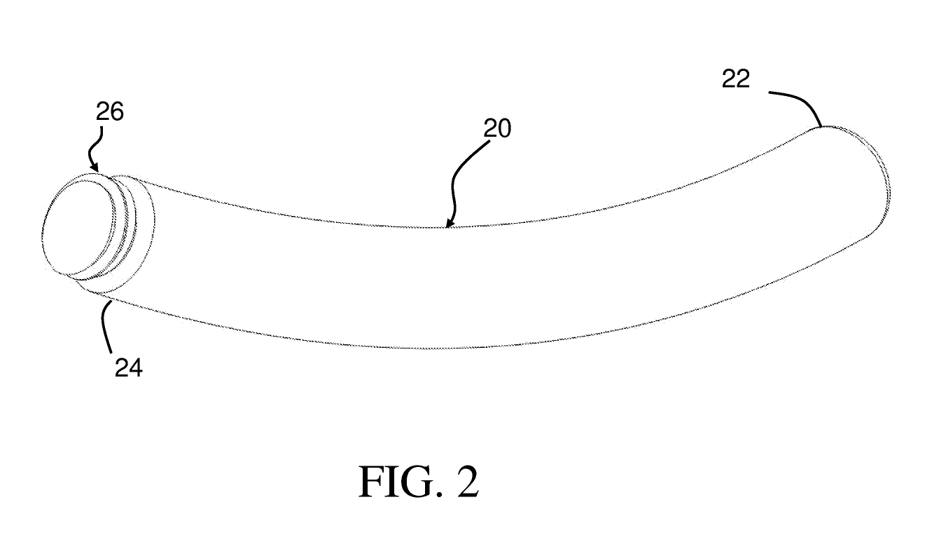

Turning now to FIG. 2, FIG. 2 is a view of the elongate member 20 of FIG. 1. The elongate member 20 includes a smooth end 22, a threaded end 24, and threads 26. The threads 26 are formed on the threaded end 24 of the elongate member 20. The threads 26 couple the end ring to the elongate member 20. The threads 26 extend along the threaded end 24 of the elongate member 20. The outer diameter of the threads 26 is equal to or less than the diameter of the elongate member 20. This allows the elongate member 20 to be placed through the receiver of the screw assembly from an inferior or superior direction (for example, in the mini-open procedure previously described), as the arms of the U-shaped channel are at least as wide as the diameter of the elongate member 20. Once the elongate member 20 is positioned in the receiver, then the end ring 30 may be delivered to the threaded end 24 of the elongate member 20 and fixed to the elongate member 20 to form the spinal rod 10.

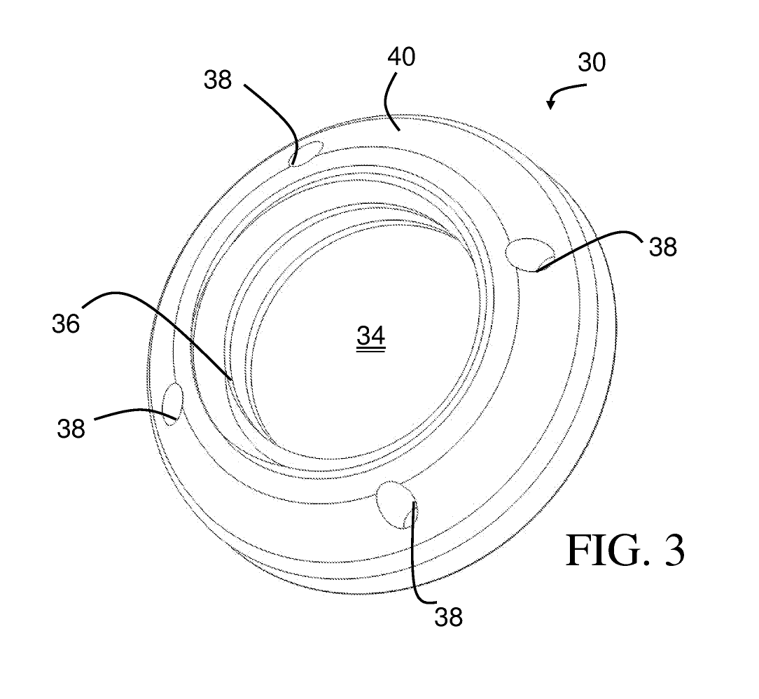

Turning now to FIG. 3, FIG. 3 is view of the end ring 30 of FIG. 1. The end ring 30 includes an aperture 34, threads 36, tool recesses 38 and a tapered surface 40. The aperture 34 receives the elongate member 20. The threads 36 of the end ring 30 mate with the threads 26 of the elongate member 20 to fix the end ring 30 to the elongate member 20. While the connection in this embodiment is threaded, other embodiments may include other connections such as a taper fit, a rivet or pinned configuration. A threaded configuration may allow the end ring 30 to be removed from the elongate member 20. Removal of the end ring 30 may be beneficial in a revision, where the threaded end of the elongate member 20 may then become a connection point for the revision.

The tapered surface 40 of the end ring 30 may be tapered to provide more clearance near the facet joint. The inner portion of the tapered surface 40 has a thickness sufficient to support the connection means (in this embodiment the threads 36) for the end ring 30 while the outer portion of the end ring 30 is sized to sufficiently capture the end ring 30 in the receiver of the screw assembly. The taper between the inner portion and outer portion of the end ring 30 may transition between these two competing requirements. The end feature may have other surfaces to create the thickness necessary to support the threads and the width necessary to capture the end feature in the receiver. For example, a star shaped end feature may provide the necessary strength and dimensions to capture the elongate member 20 within the screw assemblies.

Tool recesses 38 are located on the tapered surface 40 of the end ring 30. The tool recesses 38 are configured to allow an instrument to attach to the end ring 30 for positioning the end ring 30 on the elongate member 20 in situ. While the tool recesses 38 are radially offset around the tapered surface 40 of the end ring 30, the tool recesses 38 may be located in other orientations depending on the connection mechanism between the end ring 30 and the elongate member 20. Additionally, the recesses 38 may not be recesses, but instead be projections from the surface 40.

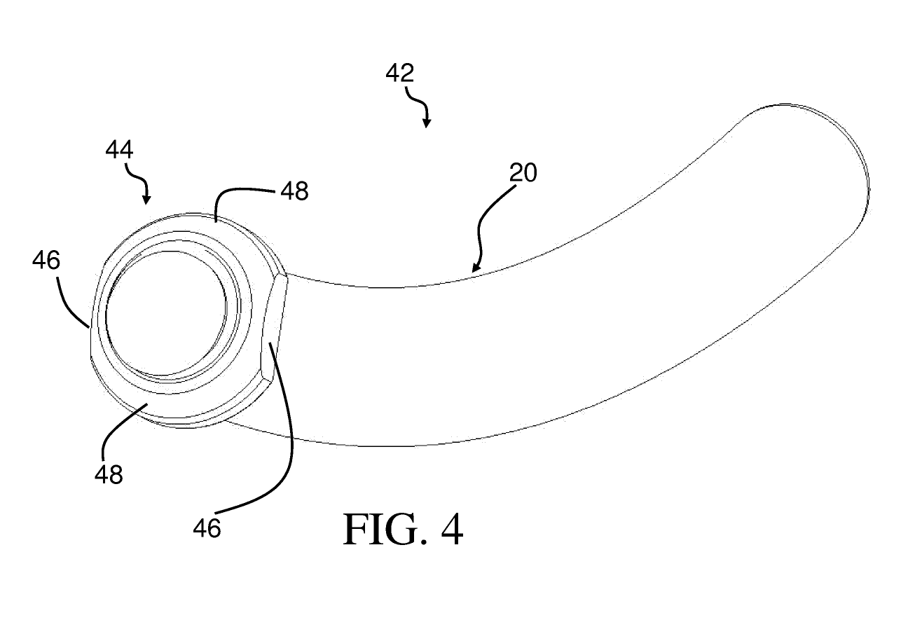

Turning now to FIG. 4, FIG. 4 shows a view of a spinal rod 42 according to another embodiment of the invention. The spinal rod 42 includes an elongate member 20 and an end feature 44. The end feature 44 has a pair of flats 46. Extending members 48 of the end feature 42 extend radially outward from the elongate member to provide sufficient width to capture the spinal rod 42 in screws. The flats 46 may present a lower profile for the spinal rod 42. Additionally, the flats 46 may provide surfaces to rotate the end feature 44 relative to the elongate member 20. In such an embodiment, tool recesses may not be necessary.

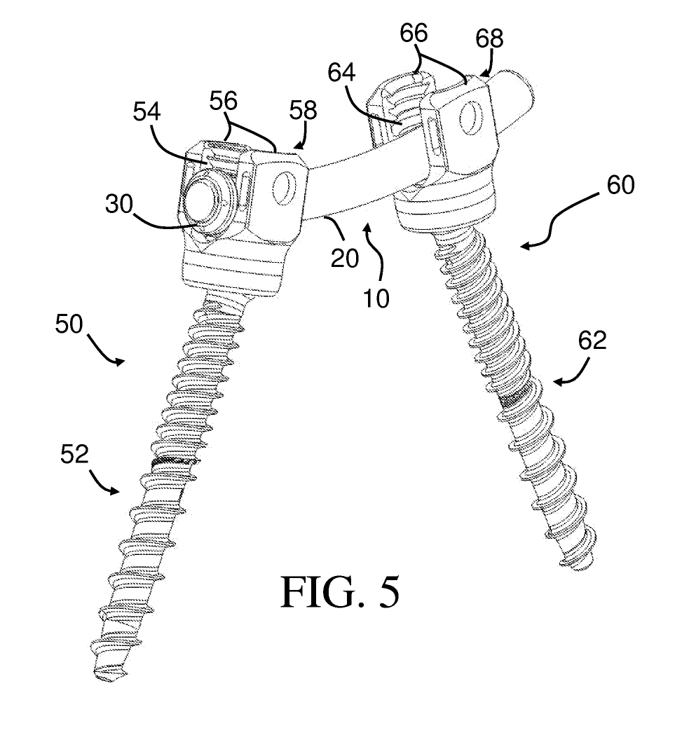

Turning now to FIG. 5, FIG. 5 is a view of the spinal rod 10 of FIG. 1 attached to two screw assemblies 50 and 60. Screw assemblies 50 and 60 include bone engaging portions 52 and 62, U-shaped channels 54 and 64, pairs of arms 56 and 66, and upper portions 58 and 68 for engaging a set screw 70. Screw assembly 50 engages the end ring 30 of the spinal rod 10 so that the end of the spinal rod 10 abuts the screw assembly 50. In a minimally invasive procedure, the elongate member 20 of the spinal rod 10 is passed through the channel 54 below the surface of the upper portion of the screw assembly 50. If screw extenders are placed on the screw assemblies 50 and 60, then the end feature glides along the outside of the channel 54 along the arms as the spinal rod 10 is directed into the channel 54.

The foregoing detailed description is provided to describe the invention in detail, and is not intended to limit the invention. Those skilled in the art will appreciate that various modifications may be made to the invention without departing significantly from the spirit and scope thereof.

Furthermore, as used herein, the terms construct and system may be interchanged. It is understood that spatial references, such as “first,” “second,” “exterior,” “interior,” “superior,” “inferior,” “anterior,” “posterior,” “central,” “annular,” “outer,” and “inner,” are for illustrative purposes only and can be varied within the scope of the disclosure.

1. A spinal rod comprising:
an elongate member having a diameter; and
an end feature configured to be selectively connected to the elongate member, the end feature having an extending member projecting radially away from the elongate member and having an outer distance of the radial extent greater than the diameter of the elongate member.

2. The spinal rod of claim 1, wherein the end feature is a ring.

3. The spinal rod of claim 1, wherein the end feature is connected to the elongate member by mating threads on the elongate member and the end feature.

4. The spinal rod of claim 3, wherein the end feature further comprises a tool recess configured to receive an insertion tool such that the end feature may be rotatably turned by the insertion tool through a connection at the tool recess.

5. The spinal rod of claim 1, wherein the elongate member is configured to be guided along a spinal column through a minimally invasive surgical procedure.

6. The spinal rod of column 5, wherein the end feature is configured to be selectively connected to the elongate member after the elongate member has been guided into position along the spinal column.

7. A spinal construct, comprising:
a spinal rod comprising:
an elongate member having a diameter; and
an end feature configured to be selectively connected to the elongate member, the end feature having an extending member projecting radially away from the elongate member and having an outer distance of the radial extent greater than the diameter of the elongate member;
a screw assembly having a U-shaped channel for receiving the elongate member and a bone engaging portion for attaching to bone, the U-shaped channel having a pair of arms extending away from the bone engaging portion, the arms configured to mate with a portion of the end feature to limit the movement of the elongate member relative to the screw assembly; and
a set screw configured to capture the elongate member to the screw assembly.

8. The spinal construct of claim 7, wherein the end feature is a ring.

9. The spinal construct of claim 7, wherein the end feature is connected to the elongate member by mating threads on the elongate member and the end feature.

10. The spinal construct of claim 9, wherein the end feature further comprises a tool recess configured to receive an insertion tool such that the end feature may be rotatably turned by the insertion tool through a connection at the tool recess.

11. The spinal construct of claim 7, wherein the elongate member is configured to be guided between the arms of the screw assembly such that the threads of the elongate member pass through the U-shaped channel.

12. The spinal construct of column 11, wherein the end feature is configured to be selectively connected to the elongate member after the elongate member has been guided into position along the spinal column.

13. The spinal construct of claim 7, wherein the end feature is configured to be selectively connected to the elongate member before the elongate member has been guided into position along the spinal column such that the end feature is guided along the outside of the arms of the U-shaped channel of the screw assembly.
